(19) United States
(12) Patent Application Publication
(10) Pub. No.: US 2015/0362509 A1
(43) Pub. Date: Dec. 17, 2015

(54) Method for Differentiating Sepsis and Systemic Inflammatory Response Syndrome (SIRS)

(71) Applicant: VANDERBILT UNIVERSITY, Nashville, TN (US)

(72) Inventor: Alison Woodworth, Nashville, TN (US)

(21) Appl. No.: 14/763,764

(22) PCT Filed: Jan. 28, 2014
(86) PCT No.: PCT/US14/13407

§ 371 Date: Jul. 27, 2015

Related U.S. Application Data
(60) Provisional application No. 61/757,393, filed on Jan. 28, 2013.

(51) Int. Cl. G01N 33/68

(52) U.S. Cl. CPC G01N 33/6893; G01N 33/6863; G01N 33/6869; G01N 33/6866

(57) ABSTRACT

A method of characterizing sepsis in a subject involves determining an amount in a biological sample from the subject of each biomarker in a biomarker panel including at least two biomarkers; and calculating a risk index or sepsis probability score using the amounts of biomarkers in the biomarker panel. Exemplary biomarker panels include TNF-alpha, interleukin-6, interleukin-10, lipopolysaccharide binding protein, and C-reactive protein; IL-1beta, procalcitonin, absolute neutrophil count, and immature granulocyte count; and Interleukin-8, GM-CSF, MCP1, and INF-gamma. The method can further involve determining one or more clinical parameters about the subject, and calculating the risk index using the amounts of the biomarkers in the biomarker panel and the clinical parameters.

### RELATED APPLICATIONS

This application claims priority from U.S. Provisional Application Ser. No. 61/757,393 filed Jan. 28, 2013, the entire disclosure of which is incorporated herein by this reference.

### GOVERNMENT INTEREST

This invention was made with government support under 1UL1RR024975 awarded by the National Institutes of Health. The government has certain rights in the invention.

### TECHNICAL FIELD

The presently-disclosed subject matter relates to differentiation of sepsis and other conditions, such as systemic inflammatory response syndrome (SIRS), in a subject.

### INTRODUCTION

Sepsis is a life-threatening condition affecting approximately 27 million people worldwide each year. Without appropriate treatment, sepsis can progress to severe sepsis and septic shock, which cause some eight million deaths each year. The risk of sepsis-related mortality increases with age and coexisting diseases such as diabetes or cancer; thus the incidence of sepsis has increased significantly. with hospitalizations and related costs doubling over the last 10 years. (Hall 2011).

Major efforts to reduce sepsis-related mortality have focused on generating consensus definitions of sepsis and related conditions and establishing guidelines for early identification and implementation of goal-directed therapy. In addition, studies have been undertaken to identify new sepsis biomarkers and therapies. Clinical studies show that mortality is significantly reduced if septic patients are identified and treated at early stages of the disease process. (Kumar 2006). However, early diagnosis is complicated by heterogeneity in clinical presentation, co-morbidities, and the scarcity of biomarkers that enable clinicians to accurately identify sepsis in early stages of disease. The recent revision and expansion of guidelines, with a deeper understanding of the pathophysiology of sepsis and availability of novel diagnostic and therapeutic tools, points to a better forecast for this severe disease in the future.

In 1991, the American College of Chest Physicians and the Society of Critical Care Medicine introduced the international consensus definitions for sepsis and the systemic inflammatory response syndrome (SIRS) as follows: SIRS: Inflammatory response to infectious and non-infectious conditions characterized by the presence of at least two of four signs: fever or hypothermia, tachypnea, tachycardia, and abnormally elevated or suppressed white blood cell count; Sepsis: SIRS+documented infection, identified by microbial culture; Severe sepsis: Sepsis+organ dysfunction; and Septic shock: Severe sepsis+hypotension and decreased peripheral perfusion.

These definitions were fundamental for better disease recognition and characterization of severity, and they served as a framework for inclusion criteria in clinical studies. Identifying patients with SIRS became so straightforward that informatics tools could be designed to rapidly identify patients. (Hooper 2012). However, the definitions had their limitations. Determining which SIRS patients were septic remained a challenge and relied heavily on clinical expertise and judgment. Many non-infectious conditions also present with SIRS, including pancreatitis, burns and post-surgery. In septic patients, infection cannot always be confirmed microbiologically.

These shortcomings were partially addressed ten years later with a revised sepsis definition that included confirmed or suspected infection in patients with SIRS, and signs of systemic inflammation due to infection. (Levy 2003). These signs include both biochemical and clinical parameters and are grouped in four categories: general, inflammatory, hemodynamic, and tissue perfusion parameters.

The Surviving Sepsis Campaign (SSC) Guidelines, first published in 2004 and revised in 2008 and 2013, are evidence-based guidelines for the management of severe sepsis and septic shock. (Dellinger 2013, Dellinger 2004, Dellinger 2012). The guidelines describe two sepsis treatment bundles, resuscitation and management, which must be implemented within hours after severe septic patients present to the emergency department. Implementation of these guidelines decreases mortality. (Levy 2010).

Both the sepsis definitions and the SSC guidelines include biochemical criteria, but they are supportive rather than diagnostic tests—because to date no single biochemical/inflammatory biomarker has had sufficient diagnostic strength to predict sepsis. SIRS criteria, pathogen identification, and clinical variables remain the cornerstone of the sepsis definition, but they are often unable to identify patients in early stages of disease. Clinical signs like the SIRS criteria are nonspecific for sepsis, and microbiological cultures can take days to produce results. There is a great need for sensitive and specific biomarkers that can identify patients in early stages of sepsis, determine disease severity and predict response to therapy.

The pathophysiology of sepsis is complex. Recent theories describe a host's global response to infection as having both pro-inflammatory and compensatory anti-inflammatory stages. (Faix 2013). The exact sequence of events is unclear. One theory supports a sequential model in which inflammation is followed by anti-inflammation, while a second model, known as the mixed antagonist response syndrome, suggests that these two occur simultaneously. Dysregulation of the inflammatory/anti-inflammatory response may contribute to sepsis-related mortality. A prolonged hyper-inflammatory response results in a storm of toxic cytokines, while an immunosuppressive stage impairs protection against pathogens. No matter the sequence, dysregulation of the immune response to an infection leads to altered coagulation and cellular activation, endothelial cell failure and cellular apoptosis, all of which contribute to metabolic alterations and multi-organ damage, and eventually death. (Femick 2007). A clear understanding of sepsis pathophysiology important for development of novel therapeutic and diagnostic tools.

Lactate, C-reactive protein (CRP), and procalcitonin (PCT) are commonly used for classification and management of septic patients. Lactate is used to assess tissue perfusion and is elevated with tissue hypoxia caused by hypoperfusion in severe sepsis and septic shock but not in early sepsis. The SSC guidelines recommend measuring lactate within three hours after sepsis is suspected; a lactate >4 mmol/L warrants fluid resuscitation. If initial concentration is above that cut-off, lactate is remeasured within a few hours to evaluate response to therapy. Patients achieving a lactate clearance >10% have better prognoses. (Arnold 2009).

CRP and PCT are both inflammatory biomarkers, widely investigated for sepsis diagnosis. (Faix 2013, Tang 2007, Pierrakos 2010). CRP is an acute-phase reactant elevated in many inflammatory conditions. PCT, the precursor of the thyroid hormone calcitonin, is also increased in the systemic inflammatory response to infection. The Food and Drug Administration-approved PCT testing is indicated in conjunction with other laboratory and clinical findings for the diagnosis of bacterial infection and sepsis in critically ill patients. Overall, most studies indicate superior clinical utility (sensitivity and specificity) of PCT over CRP for the identification of sepsis among patients with systemic inflammation. (Tang 2007, Meisner 2000, Simon 2004). The concentration of PCT correlates with severity of disease, while CRP is not helpful for stratification. (Meisner 2000).

The utility of PCT remains controversial and it is not universally adopted in clinical practice. (Tang 2007). Both CRP and PCT are listed among the inflammatory variables that serve as criteria to diagnose sepsis, but the SSC guidelines state that the ability of PCT or CRP to discriminate between non-infectious and infectious SIRS has not been demonstrated, and they issue no recommendations for utilization of either biomarker to identify infected patients among those with systemic inflammation. The SSC guidelines endorse PCT as a tool for antibiotic stewardship. (Dellinger 2013). In adults, low PCT concentrations can be used to direct cessation of antibiotics in critically ill patients; however, high PCT concentrations should not be used to intensify antibiotic therapy. (Soni 2012). The utility of PCT is still unknown in pediatric patients and neonates. PCT-guided antimicrobial therapy reduces antibiotic use without benefits in morbidity and mortality. (Soni 2012, Schuetz 2013).

In sum, lactate, PCT, and CRP are helpful markers to manage patients with suspected sepsis by providing prognostic information and guiding therapy, but they have limited diagnostic utility in sepsis and no role at the early stages of sepsis.

Many research groups are studying novel biomarkers for sepsis diagnosis and management. The studies range in quality (sample size), inclusion criteria (SIRS, all patients, shock), hospital setting (ED, ICU, hospitalized), outcomes measured (mortality, bacterial infection, distinguishing SIRS vs. sepsis), and age (adults, neonates, children), and this variation greatly confounds the interpretation of the findings. A recent literature review identified more than 3,000 published reports of 178 sepsis biomarkers, including immune cell markers, cytokines, coagulation factors, acute-phase reactants, markers of vasculo-endothelial damage, vasodilation, and organ dysfunction. (Pierrakos 2010). The authors surmise that none of the biomarkers alone had sufficient diagnostic strength to identify septic patients.

The emerging theory is that a panel of biomarkers may better predict sepsis among patients with systemic inflammation. (Pierrakos 2010). Several studies that biomarker panels have a diagnostic utility to predict sepsis that is superior to that of any single biomarker. (Gibot 2012, Andaluz-Ojeda 2012, Kofoed 2007). And measuring multiple markers is now feasible in the clinical lab, with the recent availability of multi-analyte platforms, known as multiplex platforms, in which the concentration of panels of biomarkers (protein, DNA, or RNA) is determined simultaneously. These new technologies are efficient and can potentially improve sepsis diagnosis.

The challenge to implementing these routinely is the large amount of data generated. These platforms require robust bioinformatics and statistical tools to provide easy-to-interpret information for clinicians at the bedside. Combining the results of multiple markers into a single algorithm or risk score may ease the implementation of these panels into clinical practice. (Gibot 2012, Andaluz-Ojeda 2012, Shapiro 2009). Finally, emerging technologies in microbiology, such as molecular and MALDI-TOF techniques, have altered the way microorganisms are identified in the clinical laboratory. (Skvarc 2013, DeMarco 2013). These reduce diagnostic time and improve specificity and sensitivity compared to cultures, thereby enhancing diagnosis and management of septic patients.

Early diagnosis and treatment are fundamental to reducing sepsis related morbidity and mortality. The complexity and heterogeneity of sepsis make early diagnosis challenging (See FIG. 1). No single biomarker has sufficient diagnostic strength to identify septic patients among those with systemic inflammation at early stages. Accordingly, there remains a need in the art for a reliable test to rule-in sepsis at an early stage.

### SUMMARY

The presently-disclosed subject matter meets some or all of the above-identified needs, as will become evident to those of ordinary skill in the art after a study of information provided in this document.

Methods of the presently-disclosed subject matter for characterizing sepsis in a subject involve determining an amount in a biological sample from the subject of each biomarker in a biomarker panel, and calculating a risk index using the amounts of the at least two biomarkers. Biomarker panels include at least two biomarkers, as disclosed herein. Examples of biomarker panels include: TNF-alpha, interleukin-6, interleukin-10, lipopolysaccharide binding protein, and C-reactive protein; IL-1beta, procalcitonin, absolute neutrophil count, and immature granulocyte count; or Interleukin-8, GM-CSF, MCP1, and INF-gamma.

The biological sample can include blood, plasma, and/or serum. In some embodiments, the biological sample is extracted prior to determining an amount of each biomarker in the sample. The biological sample can be from a subject, such as a human subject, having symptoms of SIRS and/or sepsis risk factors.

Embodiments of the methods also involve determining one or more clinical parameters about the subject, and calculating the risk index using the amounts of the at least two biomarkers and the clinical parameters. Clinical parameters can include the clinical parameters include demographic parameters and laboratory values, such as, respiratory rate, white blood cell count, body temperature, heart rate, systolic blood pressure, morning blood glucose, platelet count, red blood cell count, lactate, PTT, INR, bilirubin, creatinine, urine output, whether the subject is culture positive, location of the positive culture, age at SIRS alert, sex, BMI, origin of patient, APACHE II score, whether the subject received inotropics, whether the subject was receiving antibiotics, whether the subject was on a ventilator, whether the subject was treated with vasopressors, whether the subject was diagnosed with a solid tumor or hematoligical malignancies, whether the subject was receiving immunosuppressive treatment, whether the subject has cirrhosis, whether the subject has chronic renal failure, whether the subject has COPD or Asthma, whether the subject is diabetic, whether the subject was receiving chemotherapy, and whether the subject has HIV/AIDS.

In some embodiments, calculating the risk index involves applying a predetermined weight for the amount of each biomarker in the panel and each of the one or more clinical parameters; and solving one or more equations, wherein the one or more equations is based, at least in part, on the weights for the biomarker amounts and the clinical parameters. In some embodiments, the risk index is calculated using at least one algorithm. The at least one algorithm can include a logistic regression analysis and/or a probability analysis.

The risk index or sepsis probability score (SPS) can be correlated to a predicted clinical outcome. The predicted clinical outcome can be, for example, a diagnosis of sepsis, a diagnosis of early sepsis, or a diagnosis of SIRS. In some embodiments, the method can also involve recommending, modifying, and/or administering treatment, such as treatment for sepsis.

In some embodiments, the method can include determining an amount in each of a series of biological samples sample of each biomarker in the biomarker panel. The series of biological samples can include a first biological sample collected prior to initiation of a prophylaxis or treatment and a second biological sample collected after initiation of the prophylaxis or treatment. Such embodiments of the method can be useful for monitoring a subject.

In some embodiment, the method involves providing a probe for selectively binding each of the biomarkers in the biomarker panel. In this regard, the method can include measuring an amount of marker-bound probe for each of the biomarkers. In some embodiments of the method, determining the amount in the sample of the biomarkers involves use of mass spectrometry (MS) analysis, immunoassay analysis, or both. In some embodiments, the immunoassay analysis comprises an enzyme-linked immunosorbent assay (ELISA).

Embodiments of the method can also include providing an apparatus capable of detecting the biomarkers in the biomarker panel

The presently disclosed subject matter further includes systems and kits comprising probes for each of the biomarkers in a panel and/or an apparatus for detecting biomarkers in the sample and/or software for calculating risk index.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1. Complex Pathophysiology of Sepsis. Systemic infection leads to a global immune response which produces pro-inflammatory cytokines (e.g., TNF-α, INFγ, IL-1β, IL-6) and anti-inflammatory cytokines (e.g., IL-10); these promote endothelial damage, mitochondrial dysfunction, coagulation activity, and death.

FIG. 2. Sepsis, the body's response to systemic infection. Sepsis is systemic inflammatory infection (SIRS), in the presence of infection. Clinical signs and symptoms overlap between Sepsis and SIRS.

FIGS. 3A and 3B. Performance characteristics of the individual biomarkers to predict sepsis in patients with SIRS. ROC curves were generated for each of the ten markers, IL-1β, IL-6, IL-8, IL-10, GM-CSF, MCP-1, TNFα, INFy, hsCRP, and PCT (A) Using the ROC curves, cut-offs were selected at optimal sensitivity (Sens), specificity (Spec). (B) Only the ROC curves for IL-6, MCP-1, and PCT differed significant from identity (p<0.05). Only IL-1β, with a positive likelihood ratio (LR+) >5, has moderate diagnostic strength to rule in sepsis.

FIG. 4. Diagnostic utilities of multimarker models to predict sepsis. Combined analysis yields higher AUC results compared to any single marker The ROC curves for all biomarker combinations shown are significantly different from identity (p<0.05). The Akaike's information criteria (AIC) evaluation of the predictive ability of a model shows that the two marker model “best” predicts sepsis.

FIG. 5. Performance characteristics of three models to predict sepsis vs. SIRS. ROC curves were generated for demographics, vital signs and lab values (Model A), Inflammatory biomarkers (Model B), and both combined (Model AB). ΔROC=0.12 (A vs. AB) p=0.0009.

FIG. 6. Diagnostic utility logistic regression models to predict severe sepsis/septic shock. Combined analysis yields higher AUC. ΔROC=0.16 (A vs. AB) p<0.001.

FIG. 7 is a diagram illustrating the identification of patients and collection of specimens for the studies described in Example 6.

FIG. 8 is a diagram illustrating the experimental design of the studies described in Example 6.

FIG. 9 SPSs derived from Model AB are useful for risk stratification. Patients with SIRS and sepsis were grouped according to their SPS into quartiles; upper diagrams. The lower diagrams represent patient stratified by sepsis severity and grouped in the corresponding SPS quartile.

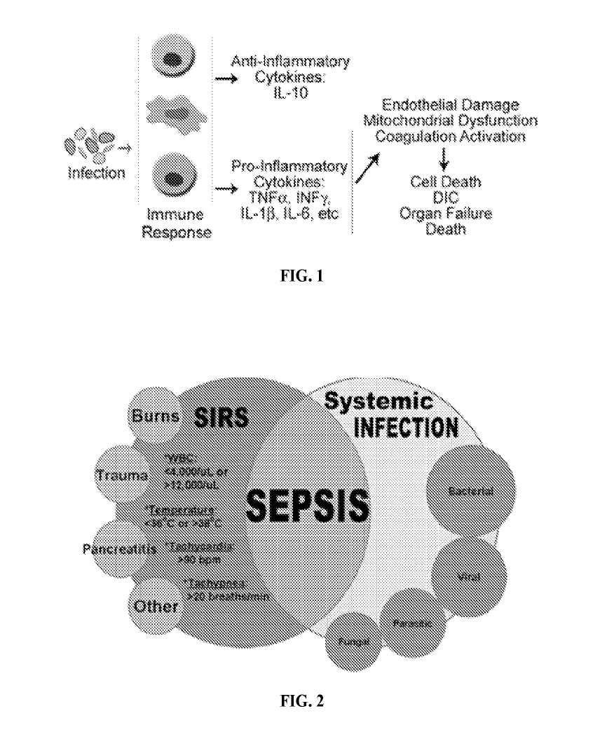

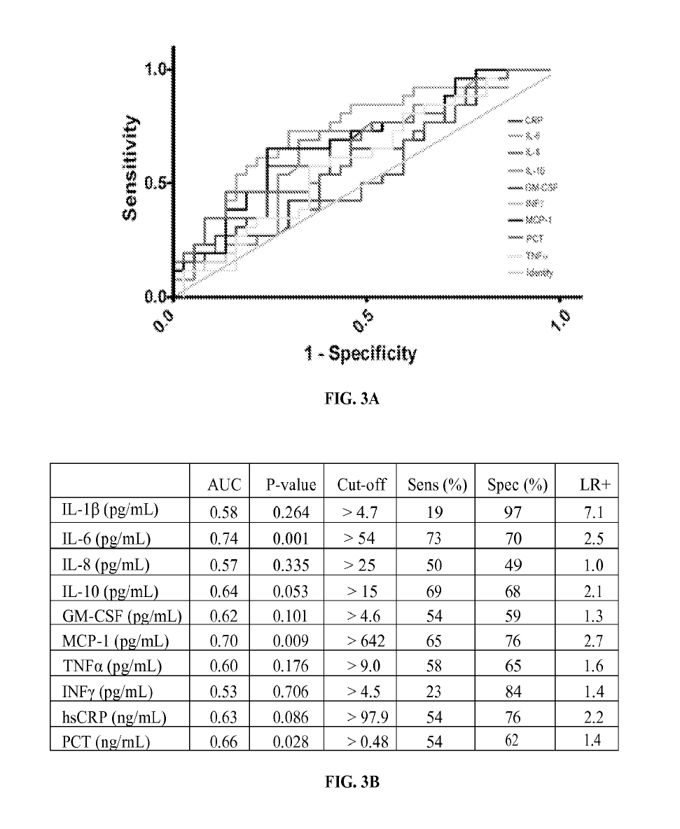

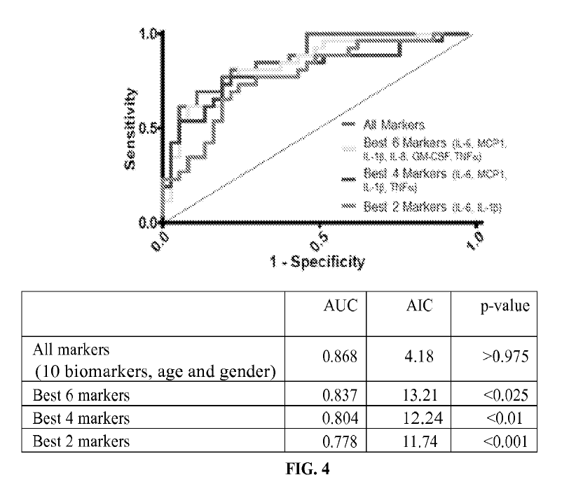

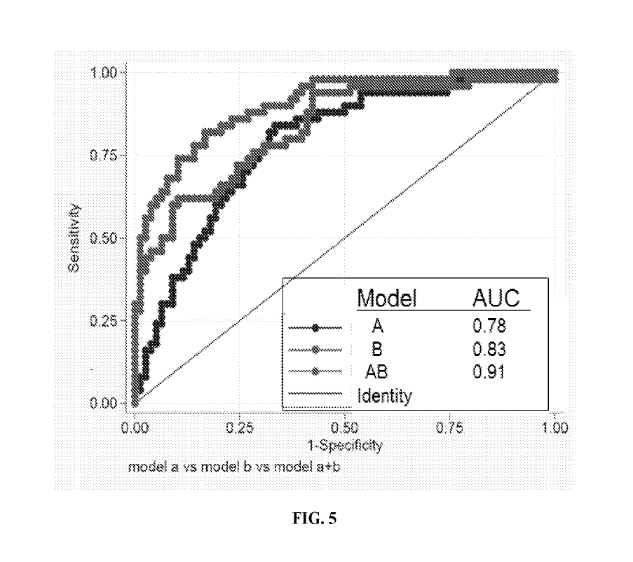

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter includes method, systems, and kits useful for characterizing sepsis, including early sepsis, in a subject, characterizing systemic inflammatory response syndrome (SIRS) in a subject, and distinguishing between SIRS and sepsis in a subject. Embodiments of the methods include providing a biological sample from the subject; determining an amount in the sample of at least two biomarkers in a biomarker panel, determining one or more clinical parameters about the subject, and calculating a risk index. The risk index can be used to predict a clinical outcome. In some embodiments, the risk index can be a sepsis probability score (SPS), wherein treatment for sepsis is recommended or administered when the SPS is above a predetermined threshold amount. For example, in some embodiments treatment for sepsis could be recommended when there is a SPS of at least about 25, 25, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95%.

In some embodiments, the biological sample can be selected from blood, plasma, or serum. In some embodiment, the biological sample is extracted. In some embodiments, the amount of each biomarker or a biomarker expression profile is compared to a reference, thereby allowing for characterization of sepsis, early sepsis, or SIRS, and/or for distinguishing between SIRS and sepsis.

In some embodiments, the biomarkers of the biomarker panel are selected from those set forth in Table 1A, Table 1B, and Table 1C. In some embodiments, the clinical parameters are set forth in Table 2A and Table 2B.

### Biomarkers

TABLE 1A

TNF-alpha
Interleukin-6 (IL-6)
Interleukin-10 (IL-10)
Lipopolysaccharide Binding Protein (LBP)
C-reactive protein (CRP)

TABLE 1B

IL-1beta
Procalcitonin (PCT)
Absolute neutrophil count
Immature granulocyte count

TABLE 1C

Interleukin-8 (IL-8)
GM-CSF
MCP1
INF-gamma

### Clinical Parameters

TABLE 2A
demographic parameters

Age at SIRS alert
Sex
BMI
Origin of patient
APACHE II Score
Did patient receive inotropics (Y/N)
Was the patient receiving antibiotics (Y/N)
Was the patient on a ventilator?
Was the patient treated with vasopressors?
Was patient diagnosed with a solid tumor or hematological malignancies?
Was the patient receiving immunosuppressive treatment?
Did patient have Cirrhosis?
Did patient have chronic renal failure?
Did patient have COPD or Asthma?
Was patient diabetic ?
Was patient receiving chemotherapy?
Did patient have HIV/AIDS?

TABLE 2B
Laboratory Values/Clinical Parameters

Respiratory Rate
White Blood Cell Count
Body Temperature
Heart rate
Systolic Blood Pressure
Morning blood glucose
Platelet Count
Red Blood Cell Count
Lactate
PTT
INR
Bilirubin
Creatinine
Urine output (total volume)
Culture positive (Y/N)
Culture positive - location

A “biomarker” is a molecule useful as an indicator of a biologic state in a subject. With reference to the present subject matter, the biomarkers disclosed herein can be polypeptides that exhibit a change in expression or state, which can be correlated to a predicted clinical outcome, e.g., characterization of sepsis, characterization of early sepsis, characterization of SIRS, differentiating between SIRS and sepsis or early sepsis. In addition, the biomarkers disclosed herein are inclusive of messenger RNAs (mRNAs) encoding the biomarker polypeptides, as measurement of a change in expression of an mRNA can be correlated with changes in expression of the polypeptide encoded by the mRNA. As such, determining an amount of a biomarker in a biological sample is inclusive of determining an amount of a polypeptide biomarker and/or an amount of an mRNA encoding the polypeptide biomarker either by direct or indirect (e.g., by measure of a complementary DNA (cDNA) synthesized from the mRNA) measure of the mRNA.

The terms “polypeptide”, “protein”, and “peptide”, which are used interchangeably herein, refer to a polymer of the 20 protein amino acids, including modified amino acids (e.g., phosphorylated, glycated, etc.), regardless of size or function. Although “protein” is often used in reference to relatively large polypeptides, and “peptide” is often used in reference to small polypeptides, usage of these terms in the art overlaps and varies. The term “peptide” as used herein refers to peptides, polypeptides, proteins and fragments of proteins, unless otherwise noted. The terms “protein”, “polypeptide” and “peptide” are used interchangeably herein when referring to a gene product and fragments thereof. Thus, exemplary polypeptides include gene products, naturally occurring proteins, homologs, orthologs, paralogs, fragments and other equivalents, variants, and fragments of the foregoing.

The terms “polypeptide fragment” or “fragment”, when used in reference to a polypeptide, refers to a polypeptide in which amino acid residues are absent as compared to the full-length polypeptide itself, but where the remaining amino acid sequence is usually identical to the corresponding positions in the reference polypeptide. Such deletions can occur at the amino-terminus or carboxy-terminus of the reference polypeptide, or alternatively both.

Sepsis is systemic inflammatory infection (SIRS), in the presence of infection. Early sepsis occurs at any point in time in the sepsis pathobiological process prior to development of overt clinical symptoms of severe sepsis and/or septic shock (i.e. organ failure and/or hypotension). Sever sepsis is sepsis in combination with organ dysfunction, and is associated with about 25-30% mortality. Septic Shock is sever sepsis in combination with hypotension and is associated with about 40-70% mortality.

In some embodiments of the presently-disclosed subject matter, the method includes determining an amount in a biological sample from the subject of each biomarker in a biomarker panel, wherein the biomarker panel comprises at least two of the biomarkers as set forth in Tables 1A, 1B, and 1C; and calculating a risk index using the amounts of the at least two biomarkers. The method can also include determining one or more clinical parameters about the subject (e.g., as set forth in Tables 2A and 2B), and calculating the risk index using the amounts of the at least two biomarkers and the clinical parameters.

Calculating the risk index can involve comparing the amounts of the at least two biomarkers to references. Calculating the risk index can involve applying a predetermined weight for the amount of each biomarker in the panel and each of the one or more clinical parameters; and solving one or more equations, wherein the one or more equations is based, at least in part, on the weights for the biomarker amounts and the clinical parameters. In some embodiments, the risk index is calculated using at least one algorithm, e.g., a logistic regression analysis of references, probability analysis of references, etc. In some embodiments, it can be desirable to select references for the at least one algorithm based on a particular patient population, e.g., geographic, demographic, IUD patients, ED patients, etc. The risk index can be correlated to a predicted clinical outcome, e.g., diagnosis of sepsis, a diagnosis of early sepsis, or a diagnosis of SIRS, exclusion from identification of having sepsis, etc.

The “reference” can include, for example, amounts of biomarkers in one or more samples from one or more individuals associated with early sepsis, sepsis, SIRS, and/or without a condition associated with inflammation and/or infection. In some embodiments, the reference can include a standard sample. Such a standard sample can be a reference that provides amounts of biomarkers at levels considered to be control, early sepsis, sepsis, or SIRS levels. For example, a standard sample can be prepared to mimic the amounts or levels of the biomarkers in one or more samples (e.g., an average of amounts or levels from multiple samples) from one or more individuals of confirmed clinical status. In some embodiments, the reference can include data. Data, when used as a reference, can comprise compilations of data, such as may be contained in a table, chart, graph, e.g., standard curve, or database, which provides amounts or levels of biomarkers considered to be control, early sepsis, sepsis, or SIRS levels. Such data can be compiled, for example, by obtaining amounts or levels of biomarkers in one or more samples (e.g., an average of amounts or levels from multiple samples) from one or more individuals of confirmed clinical status.

In this regard, data can include biomarker data and clinical parameter data, associated with at least one algorithm, e.g., logistic regression analysis, probability analysis. Such data and/or at least one algorithm can be associated with software, which can be used to calculate a risk index. For example, amounts in the sample of biomarkers in the biomarker panel can be determined, and a risk index or sepsis probability score can be calculated, e.g., using a software to calculate the risk index using at least one algorithm. The at least one algorithm can include a logistic regression analysis and probability analysis of data (references). Such data points can be selected, in some embodiments, based on a particular patient population, e.g., geographic, dempograpic, etc., expanded access to further data points as such data is gathered, validated. In some, embodiments, such software could be accessed from a remote server, and a report including a risk index, sepsis probability score, associated clinical outcome, and/or recommended treatment could be provided.

Characterizing, as in characterizing sepsis in a subject, can include providing a diagnosis, prognosis, and/or theranosis of a subject, such as a subject identified as having SIRS symptoms or sepsis risk factors.

“Making a diagnosis” or “diagnosing,” as used herein, are further inclusive of making a prognosis, which can provide for predicting a clinical outcome (with or without medical treatment), selecting an appropriate treatment (or whether treatment would be effective), or monitoring SIRS or potential sepsis. Diagnostic testing that involves treatment, such as treatment monitoring or decision making can be referred to as “theranosis.” Further, in some embodiments of the presently disclosed subject matter, multiple determinations of biomarker levels/clinical parameters over time can be made to facilitate diagnosis (including prognosis), evaluating treatment efficacy, and/or progression of SIRS or potential sepsis. A temporal change in one or more levels/concentrations/parameters can be used to predict a clinical outcome, monitor the progression of the condition, and/or efficacy of administered therapies.

The phrase “determining the prognosis” as used herein refers to methods by which the skilled artisan can predict the course or outcome of a condition in a subject. The term “prognosis” can refer to the ability to predict the course or outcome of a condition with up to 100% accuracy, or predict that a given course or outcome is more or less likely to occur. The term “prognosis” can also refer to an increased probability that a certain course or outcome will occur; that is, that a course or outcome is more likely to occur in a subject when compared to individuals in a comparator group. For example, in individuals exhibiting a higher risk index than a reference, the chance of a given outcome (e.g., sepsis) may be very high. In certain embodiments, a prognosis is about a 5% chance of a given expected outcome, about a 7% chance, about a 10% chance, about a 12% chance, about a 15% chance, about a 20% chance, about a 25% chance, about a 30% chance, about a 40% chance, about a 50% chance, about a 60% chance, about a 75% chance, about a 90% chance, or about a 95% chance. In some embodiments the % chance is associated with a relative risk of sepsis, for example.

The skilled artisan will understand that associating a prognostic indicator with a predisposition to an adverse outcome can be performed using statistical analysis. Statistical significance is often determined by comparing two or more populations, and determining a confidence interval and/or a p value. See, e.g., Dowdy and Wearden, Statistics for Research, John Wiley & Sons, New York, 1983, incorporated herein by reference in its entirety. Exemplary confidence intervals of the present subject matter are 90%, 95%, 97.5%, 98%, 99%, 99.5%, 99.9% and 99.99%, while exemplary p values are 0.05, 0.025, 0.02, 0.01, 0.005, 0.001, and 0.0001. When performing multiple statistical tests, p values can be corrected for multiple comparisons using techniques known in the art.

In some embodiments, the subject is excluded from an identification of having sepsis. In some embodiments, the subject is identified as having sepsis. In some embodiments, the subject is identified as having early sepsis. In some embodiments, methods of the presently-disclosed subject matter further include selecting a treatment or modifying a treatment provided to the subject.

In some embodiments, an apparatus for detecting the biomarkers in the sample is provided and used to conduct the characterization method. In some embodiments, a probe for selectively binding each of the biomarkers in the biomarker panel can be provided and used to conduct the characterization method. In some embodiments of methods in which a probe is provided for selectively binding each of the biomarkers in the biomarker panel, the method can also include measuring an amount of marker-bound probe for each of the biomarkers.

In this regard, the presently disclosed subject matter further includes kits and devices useful for detecting and/or determining expression levels of at least two biomarkers in a biological sample. In some embodiments, the kit includes a reagent to carry out a method as disclosed herein. In some embodiments, a kit provided in accordance with the presently-disclosed subject matter includes a probe for each of the biomarkers in a panel comprising 2, 3, 4, 5, 6, 7, 8, or 9 of the biomarkers as set forth herein. The presently-disclosed subject matter further includes systems including an apparatus for detecting the biomarkers in the sample is provided and used to conduct the characterization method, and/or a software system for calculating risk index and/or clinical outcome, and/or probes (or a kit containing such probes) for selectively binding each of the biomarkers in the panel.

In some embodiments, methods disclosed herein further involve determining the amount in the sample of the biomarkers using mass spectrometry (MS) analysis, immunoassay analysis, or both. In some embodiments, the immunoassay analysis comprises an enzyme-linked immunosorbent assay (ELISA).

In some embodiments of the presently-disclosed subject matter a method for monitoring SIRS and/or sepsis in a subject is provided, which involves providing a series of biological samples over a time period from the subject; determining an amount in each sample of each biomarker in a biomarker panel, wherein the biomarker panel comprises 2, 3, 4, 5, 6, 7, 8, or 9 of the biomarkers as set forth herein; determining one or more clinical parameters about the subject, optionally associated with a time point for each of the series of biological samples; and calculating a risk index. In some embodiments, the series of biological samples comprises a first biological sample collected prior to initiation of a prophylaxis or treatment and a second biological sample collected after initiation of the prophylaxis or treatment. In some embodiments, the series of biological samples comprises a first biological sample collected prior to onset of the SIRS and/or sepsis and a second biological sample collected after the onset of the SIRS and/or sepsis.

In some embodiments, determining an amount in a sample from the subject of each biomarker in a biomarker panel comprises determining a biomarker expression profile for a biomarker panel including at least two biomarkers as disclosed herein.

Further with respect to the methods of the presently disclosed subject matter, a preferred subject is a vertebrate subject. A preferred vertebrate is warm-blooded; a preferred warm-blooded vertebrate is a mammal A mammal is most preferably a human. As used herein, the term “subject” includes both human and animal subjects. Thus, veterinary therapeutic uses are provided in accordance with the presently disclosed subject matter.

As such, the presently disclosed subject matter provides for the diagnosis of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including race horses), poultry, and the like.

In certain instances, nucleotides and polypeptides disclosed herein are included in publicly-available databases, such as GENBANK® and SWISSPROT. Information including sequences and other information related to such nucleotides and polypeptides included in such publicly-available databases are expressly incorporated by reference. Unless otherwise indicated or apparent the references to such publicly-available databases are references to the most recent version of the database as of the filing date of this Application.

While the terms used herein are believed to be well understood by one of ordinary skill in the art, definitions are set forth herein to facilitate explanation of the presently-disclosed subject matter.

Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

The presently-disclosed subject matter is further illustrated by the following specific but non-limiting examples. The following examples may include compilations of data that are representative of data gathered at various times during the course of development and experimentation related to the present invention.

### EXAMPLES

### Example 1

Studies were conducted to identify a panel of biomarkers that accurately detects sepsis in ICU patients with SIRS. Plasma samples (n=169) collected from ICU patients on the first day they had SIRS (identified though an automated electronic medical record scan) were included. Of these, 67 had sepsis and 102 had non-infectious SIRS. Concentrations of 5 markers, CRP, LBP, IL-6, IL-10, and TNFα were determined by immunoassay on the Siemens Immulite 1000. ROC analysis for each biomarker was used to generate area under the curve and select optimal cut-offs. The ability to predict sepsis at optimal cut-offs was assessed for single and combinations of biomarkers.

Areas under the curve for the individual biomarkers were 0.76, 0.72, 0.78, 0.73, and 0.74 for CRP, TNFα, IL-6, IL-10, and LBP, respectively (p<0.0001 for all). Optimal cut-offs to identify sepsis were: CRP, 87 mg/dL (75% sensitivity, 72% specificity); TNFα, 22 pg/mL (52% sens, 82% spec); IL-6, 58 pg/mL (76% sens, 74% spec); IL-10, 12.7 pg/mL (60% sens, 84% spec); LBP, 13.8 mcg/mL (84% sens, 63% spec). Several biomarker panels showed excellent diagnostic strength to rule-in sepsis: positive likelihood ratios for marker combinations were 33.5 for all 5 markers, as well as TNFα, IL-6, IL-10, and LBP, and 32.0 for the combination of CRP, TNFa, IL-6, and IL-10 (all with positive predictive values of 96%). Patients positive for all 5 markers within 5 days of meeting SIRS criteria were more likely to die (OR=3.2) while those negative for all markers had an excellent prognosis (OR=0.04). The combination of biomarkers, CRP, TNFa, IL-6, IL-10, and LBP, shows robust diagnostic strength to rule-in sepsis and predict mortality in critically ill patients with SIRS.

### Example 2

Studies were conducted to identify a panel of biomarkers that accurately detects sepsis in ICU patients with SIRS. Plasma samples (n=63) collected from ICU patients on the first day they had SIRS (identified though an automated electronic medical record scan) were included. Of these, 26 had culture confirmed sepsis and 37 no bacterial infection within ±3 days of specimen collection. Concentrations of 8 cytokines IL-6, IL-8, IL-10, MCP-1, GM-CSF, TNFα, and INF-γ were determined by simultaneous multiplex analysis on the Luminex platform. C-reactive protein (CRP) was measured on a Roche Integra, and Procalcitonin (PCT) measured by Brahms Kryptor.

ROC curves generated for each cytokine gave areas under the curve of 0.58 (p=0.26) for IL-1β; 0.74 (p=0.001) for IL-6; 0.57 (p=0.34) for IL-8; 0.64 (p=0.05) for IL-10; 0.62 (p=0.10) for GM-CSF; 0.70 (p=0.009) for MCP-1; 0.60 (p=0.18) for TNFα; 0.53 (p=0.71) for INFγ; 0.63 (p=0.086) for CRP; 0.66 (p=0.028) for PCT. Further analysis was restricted to analytes with significant (P<0.05) curves. Cut-offs representing optimal sensitivity and specificity were 54 pg/mL, 15 pg/mL, 642 pg/mL, 97.9 ng/mL, and 0.48 ng/mL for IL-6, IL-10, MCP-1, CRP, and PCT, respectively. At these cut-offs, The diagnostic accuracies (sensitivity, specificity, and positive likelihood ratio (LR+)) for individual analytes to predict sepsis were: 73%, 70%, 2.5 (IL-6); 69%, 68%, 2.1 (IL-10); 65%, 76%, 2.7 (MCP-1); 54%, 76%, 2.2 (CPR); and 54%, 62%, 1.4 (PCT). Combining certain analytes yielded improved diagnostic strength. Patients with positive results for both IL-6 and MCP-1, IL-10 and MCP-1, and IL-6, IL-10, and MCP-1 were more likely to have sepsis LR+=3.6, 3.1, and 3.3 respectively. IL-6, IL-10, MCP-1, CRP, and PCT alone show a weak ability to identify sepsis in a population of patients with SIRS. The combination of IL-6 and MCP-1 was best able to predict sepsis.

### Example 3

Studies were conducted, which included 63 patients that were electronically identified by the Vanderbilt Listening Application. Medical ICU patients' electronic medical records are continuously monitored for SIRS criteria. Records meeting criteria in a 24 hr window automatically generate a physician alert. Thirty seven (37) patients were diagnosed with non-infectious SIRS (controls) and 26 with sepsis (cases), based on chart review. Sepsis cases must have had positive bacterial cultures within 72 hr of Alert for inclusion in the study. All patient samples were residual lithium-heparin plasma collected for physician-ordered metabolic panels from ICU patients on the day of the alert, and stored at 80° C. until analysis. IRB-approval was obtained.

Cytokine Assays: Eight cytokines: IL-1β, IL-6, IL-8, IL-10, GM-GSF, INFγ, MCP-1, and TNFα, were measured on the Luminex xMap® multiplex platform using the human cytokine immunoassay panel (Millipore).

High-sensitivity C-reactive Protein Assay: hsCRP was measured by a particle enhanced turbidimetric immunoassay on the Roche Integra®.

Procalcitonin Assay: PCT was measured using the B.R.A.H.M.S. Kryptor® PCT assay. This is a quantitative immunoassay using Time Resolved Amplified Cryptate Emission (TRACE) technology.

Statistics: All statistical analysis was performed using R environment 2.13.0 and logistical regression was performed with the “Design” package. Logistical regression was performed using stepwise and reverse methods, independently. All numerical values were transformed using the log function for regression analysis. ROC curves were generated and plotted using GraphPad Prism 5.04.

Demographics and Biomarker Results by Sepsis Status

| | SIRS | Sepsis | |
|---|---|---|---|
| | (n = 37) | (n = 26) | p-value* |
| Demographics | | | |
| Male Gender [% (n)] | 57 (21) | 42 (11) | 0.26 |
| Median age at sample | 60 | 58 | 0.75 |
| collection - years (mean ± SD) | (57 ± 21) | (56 ± 15) | |
| Median length of admission - | 6 | 15.5 | 0.004 |
| days (mean ± SD) | (8.7 ± 7.2) | (24.5 ± 30.5) | |
| Death within 28 days of collection | 11 (4) | 15 (4) | 0.59 |
| [% (n)] | | | |
| Median Biomarker Concentration | | | |
| IL-1β (pg/mL) | 0.17 | 0.17 | 0.027 |
| IL-6 (pg/mL) | 20.5 | 126.9 | <0.001 |
| IL-8 (pg/mL) | 25 | 24 | 0.34 |
| IL-10 (pg/mL) | 6.9 | 24 | 0.047 |
| GM-CSF (pg/mL) | 4.5 | 6.4 | 0.077 |
| MCP-1 (pg/mL) | 360 | 807 | 0.016 |
| TNFα (pg/mL) | 7.3 | 10.7 | 0.17 |
| INFγ (pg/mL) | 0.0 | 0.0 | 0.6 |
| hsCRP (ng/mL) | 42 | 100 | 0.094 |
| PCT (ng/mL) | 0.287 | 0.553 | 0.027 |

*All comparisons used the Wilcoxon test except age and death comparisons were by Pearson test.

Concentrations of IL-6, IL-1β, IL-10, MCP-1, and PCT were significantly different between critically ill patients with sepsis and SIRS. Alone only IL-1β showed more than minimal ability to predict sepsis. Stepwise logistic regression analysis allowed for generation of several multimarker models that all had higher AUCs than any single marker. AIC evaluation of the models demonstrated that in this population a two marker panel containing IL-6 and IL-10 was the most predictive of sepsis, p<0.001. Adding markers like cytokines, CRP, PCT and/or demographic information (age, gender) to the panel are contemplated to improve its ability to predict sepsis.

### Example 4

Analytes modeled for this pilot study included: IL-6, IL-10, LBP, CRP and TNFα. Additionally modeled data included sex and age of the patient. There were 169 total patients included in this study, 67 of which were classified as septic.

| | age | IL-6 | IL-10 | TNFα | CRP | LBP |
|---|---|---|---|---|---|---|
| Min | 18 | 2 | 1 | 1.72 | 0.3 | 1.74 |
| 1st | 43 | 17 | 2.28 | 9.78 | 15.2 | 9 |
| Quarter | | | | | | |
| Median | 57 | 49.4 | 5.25 | 14.9 | 71.8 | 16.2 |
| Mean | 54.63 | 1960.9 | 171.9 | 25.54 | 104.7 | 23.04 |
| 3rd | 67 | 173 | 17.7 | 23.7 | 166.8 | 32.2 |
| Quarter | | | | | | |
| Max | 100 | 171000 | 8700 | 208 | 469 | 98.4 |
| total at/ | | 4 (2.4%) | 9 (5.4%) | 0 (0%) | 1 (0.6%) | 0 (0%) |
| below | | | | | | |
| sens. | | | | | | |
| Septic | | 0 (0%) | 1 (0.6%) | 0 (0%) | 0 (0%) | 0 (0%) |
| w/ | | | | | | |
| values | | | | | | |
| at/ | | | | | | |
| below | | | | | | |
| sens | | | | | | |

| | ln(age) | ln(IL-6) | ln(IL-10) | ln(TNFα) | ln(CRP) | ln(LBP) |
|---|---|---|---|---|---|---|
| Min | 2.89 | 0.6931 | 0 | 0.5423 | −1.204 | 0.5539 |
| 1st | 3.761 | 2.833 | 0.8242 | 2.28 | 2.721 | 2.197 |
| Quarter | | | | | | |
| Median | 4.043 | 3.9 | 1.6580 | 2.701 | 4.274 | 2.785 |
| Mean | 3.934 | 4.183 | 2.1560 | 2.828 | 3.767 | 2.822 |
| 3rd | 4.205 | 5.1530 | 2.874 | 3.165 | 5.117 | 3.472 |
| Quarter | | | | | | |
| Max | 4.605 | 12.05 | 9.071 | 5.338 | 6.151 | 4.589 |

| Sepsis | Sex | Death |
|---|---|---|
| Y: 67 | F: 94 | N: 126 |
| N: 102 | M: 75 | Y: 43 |

A summary of the descriptive statistics for each marker is listed above. Due to the various sizes of the range for each marker, the reported values were all transformed using the natural log (ln) function and reported above.

Logistical regression models were studied in both a reverse and stepwise manner. The results are described below for each. The standard form of a logistical regression equation is p(“+” outcome)=1/(1+e−β) and β=β0+β1X1+β2X2+ . . . .

Stepwise LOGISTICAL REGRESSION ANALYSIS. Stepwise logistical regression was performed by fitting combinations of the reported analytes to a logistical model and comparing the p-values for each variable in a forward and reverse manner. No models incorporating more than three variables were found to have variables with individual p-values <0.1

Single Analyte Model Analysis

| | | | | | AIC |
|---|---|---|---|---|---|
| Analyte | AUC | p-value | Likelyhood | Coefficients (b0, b1) | (χ² scale) |
| Age | 0.5 | 0.3897 | 0.74 | −1.8147, 0.3539 | −1.16 |
| IL-6 | 0.783 | 0 | 43.78 | −2.9899, 0.6013 | 41.78 |
| IL-10 | 0.745 | 0 | 34.98 | −1.743, 0.6113 | 32.98 |
| CRP | 0.77 | 0 | 41.94 | −3.6508, 0.7912 | 39.94 |
| LBP | 0.757 | 0 | 32.4 | −3.984, 1.227 | 30.4 |
| TNFa | 0.723 | 0 | 29.1 | −3.797, 1.182 | 27.1 |
| Sex = | 0.534 | 0.3862 | 0.75 | −0.3001, −0.2753 | −1.25 |
| M | | | | | |

Two Analyte Model Analysis

| Analytes | AUC | p-value | Likelyhood | Coefficients (b0, b1, b2) | AIC (χ² scale) |
|---|---|---|---|---|---|
| IL-6, IL-10 | 0.797 | 0 | 52.51 | −3.1787, 0.4664, 0.3648 | 48.51 |
| IL-6, CRP | 0.817 | 0 | 56.85 | −4.4164, 0.4096, 0.5420 | 52.85 |
| IL-6, LBP | 0.808 | 0 | 52.12 | −4.6139, 0.4599, 0.7570 | 48.12 |
| IL-6, TNFa | 0.803 | 0 | 49.23 | −4.3213, 0.4971, 0.6240 | 45.23 |
| IL-10, LBP | 0.824 | 0 | 58.26 | −5.0343, 0.5902, 1.1486 | 54.26 |
| IL-10, TNFa | 0.775 | 0 | 43.06 | −3.4920, 0.4559, 0.7327 | 39.06 |
| IL-10, CRP | 0.837 | 0 | 67.84 | −4.913, 0.605, 0.776 | 63.84 |
| CRP, TNFa | 0.803 | 0 | 53.34 | −5.5705, 0.6697, 0.8402 | 49.34 |
| LBP, TNFa | 0.794 | 0 | 47.41 | −6.0242, 0.9878, 0.955 | 43.41 |

Three Analyte Model Analysis

| Analyte | AUC | p-value | Likelyhood | Coefficients (b0, b1, b2, b3) | AIC (χ² scale) |
|---|---|---|---|---|---|
| IL-6, CRP, | 0.824 | 0 | 60.61 | −5.4958, 0.3289, 0.5196, 0.5288 | 54.61 |
| TNFa | | | | | |
| IL-6, IL-10, | 0.833 | 0 | 63.52 | −5.2544, 0.2789, 0.4485, 0.9153 | 57.52 |
| LBP | | | | | |
| IL-10, LBP, | 0.829 | 0 | 61.52 | −5.9765, 0.4918, 1.0594, 0.4941 | 55.52 |
| TNfa | | | | | |
| IL-6, LBP, | 0.816 | 0 | 56.64 | −5.7861, 0.3613, 0.7374, 0.5752 | 50.64 |
| TNFa | | | | | |

Reverse Logistical Regression Analysis. Reverse regression analysis was performed by fitting a logistical regression model incorporating all variables. Analytes with the highest p-value were removed individually until all incorporated variables had a p-value <0.1. The order of model development is attached below.

| | | | | AIC |
|---|---|---|---|---|
| Analytes | AUC | p-value | Likelyhood | (χ² scale) |
| All markers | 0.847 | 0 | 71.01 | 57.01 |
| IL-6, IL-10, CRP, LBP, | 0.847 | 0 | 71.01 | 58.75 |
| TNFa, Sex | | | | |
| IL-6, IL-10, CRP, TNFa, Sex | 0.847 | 0 | 70.98 | 60.74 |
| IL-6, IL-10, CRP, TNFa | 0.845 | 0 | 70.72 | 62.72 |
| IL-6, IL-10, CRP | 0.843 | 0 | 70.02 | 64.02 |
| IL-10, CRP | 0.837 | 0 | 67.84 | 63.84 |

All Markers. β−5.38646+−0.02620*(ln(age))+0.18215*(ln(IL-6))+0.46368*(ln(IL-10))+0.656368*(ln(CRP))+0.07777*(ln(LBP))+0.22760*(ln(TNFa))−0.21105*(sex(M=1))

Best 6 Markers. β−5.48987+0.18186*(ln(IL-6))+0.46309*(ln(IL-10))+0.60753*(ln(CRP))+0.08127*(ln(LBP))+0.22655*(ln(TNFα))−0.21395*(sex(M=1))

Best 5 Markers. β−5.4169+0.1832*(ln(IL-6))+0.4631*(ln(IL-10))+0.6454*(ln(CRP))+0.2258*(ln(TNFα))−0.2034*(sex(M=1))

Best 4 Markers. β−5.5301+0.1711*(ln(IL-6))+0.4673*(ln(IL-10))+0.6480*(ln(CRP))+0.2258*(ln(TNFα))

Best 3 Markers. β−5.0820+0.1939*(ln(IL-6))+0.5035*(ln(IL-10))+0.6670*(ln(CRP))

Best 2 Markers. β−4.913+0.605*(ln(IL-10))+0.776*(ln(CRP))

### Example 5

Studies were conducted to determine the diagnostic utility of logistic regression models consisting of inflammatory biomarkers, demographics and/or common laboratory values to identify sepsis in patients with SIRS and/or another sepsis risk factor.

Materials and Methods.

Specimen Selection and Collection: Residual plasma specimens from 128 Emergency Department (ED) patients with SIRS and/or another sepsis risk factor (hypotension (SBP<100), altered mental status, immunodeficiency, advanced age, or hyperglycemia without diabetes) sent to the Vanderbilt University Medical Center (VUMC) Core Laboratory for routine BMP analyses were utilized. The electronic medical records of study subjects were reviewed to obtain baseline demographics, routine lab values, and vital signs. IRB approval was obtained for this study.

Biomarker Analysis: TNFα, IL-6, IL-10, CRP and LBP were quantitated using the Siemens Immulite 1000 (Siemens Healthcare Diagnostics, Inc.)

Models:

Model A—White blood cell count, Respiratory rate, Morning blood glucose, Heart rate, Temperature, Platelets, and Demographics—Age, Sex, BMI;

Model B: TNFα, IL-6, IL-10, CRP, and LBP (inflammatory biomarkers);

Model AB: Both Models A and B combined.

Adjudication: Diagnoses were blindly adjudicated by 2 ICU physicians as follows: non-infectious SIRS: 78, Sepsis: 6, Severe Sepsis: 12, Septic Shock: 32.

Statistics: The effect of inflammatory biomarkers on outcomes in SIRS patients was assessed using logistic regression. The dependent variable in these regressions was either the development of sepsis, or the development of severe sepsis or shock. Three logistic regression models were generated for each outcome (above) using Stata Software (StataCorp). The independent covariates of these models were the baseline demographics and common laboratory values alone (A), the inflammatory biomarkers alone (B), and a combined model with all covariates (AB). Akaike information criterion (AIC) was used to measure the goodness of fit of the models.

For each model, receiver operating curves (ROCs) were generated based on the model's linear predictor. The area under each ROC curve (AUC) was derived to estimate the ability of each model to predict the outcome of interest. AUCs for individual biomarkers were generated using GraphPad Prism 5.0 (GraphPad Software).

Patient Demographics

| Clinical Variable | | SIRS | Sepsis | P-Value^{#} |
|---|---|---|---|---|
| Total Patients n = 128 (% total) | | 78 (60) | 50 (40) | |
| Gender | Male | 49 (38) | 20 (16) | 0.02 |
| | Female | 29 (23) | 30 (23) | |
| Race | Caucasian | 59 (46) | 41 (32) | 0.71 |
| | AA | 16 (13) | 8 (6) | |
| | Other | 3 (2) | 1 (1) | |

| | | | |
|---|---|---|---|
| Age (yrs) [mean +/− SD] | 56 +/− 17 | 60 +/− 15 | 0.25 |
| BMI [Mean +/− SD] | 28 +/− 7.9 | 29 +/− 7.0 | 0.25 |

^{#}The P value for gender, race, age, and BMI was generated using Fishers exact test or chi square test.

Results

Diagnostic Utility of Lab values, biomarkers
and vital signs to predict sepsis

| | Sepsis vs. No Sepsis | | |
|---|---|---|---|
| Predictor | AUC | 95% CI | P value |
| TNFα | 0.80 | [0.72-0.87] | <0.001 |
| LBP | 0.80 | [0.72-0.87] | <0.001 |
| CRP | 0.69 | [0.60-0.78] | <0.001 |
| IL-6 | 0.83 | [0.76-0.9] | <0.001 |
| IL-10 | 0.72 | [0.63-0.81] | <0.001 |
| WBC | 0.63 | [0.52-0.73] | 0.01 |
| Temp | 0.74 | [0.65-0.84] | <0.001 |
| Resp Rate | 0.62 | [0.51-0.72] | 0.02 |
| Glucose | 0.50 | [0.38-0.6] | 0.99 |
| Platelet Ct | 0.59 | [0.49-0.7] | 0.07 |
| BMI² | 0.56 | [0.46-0.66] | 0.22 |

The AUC and P values were generated using Graph pad Prism 5.0 software.

The performance characteristics of Models A, B and AB to predict sepsis vs. SIRS were assessed (FIG. 5, and table below). ROC curves were generated for demographics, vital signs and lab values (Model A), Inflammatory biomarkers (Model B), and both combined (Model AB). ΔROC=0.12 (A vs. AB) p=0.0009.

Diagnostic Utility of Lab values, biomarkers and vital
signs to differentiate Patients with SIRS and early
sepsis from severe sepsis and septic shock.

| | Early/No Sepsis vs. | | |
|---|---|---|---|
| | Severe Sepsis and Shock | | |
| Predictor | AUC | 95% CI | P value |
| TNFα | 0.81 | [0.74-0.89] | <0.001 |
| LBP | 0.81 | [0.74-0.88] | <0.001 |
| CRP | 0.71 | [0.62-0.8] | <0.001 |
| IL-6 | 0.84 | [0.77-0.91] | <0.001 |
| IL-10 | 0.74 | [0.65-0.83] | <0.001 |
| WBC | 0.62 | [0.51-0.73] | 0.2 |
| Temp | 0.75 | [0.65-0.85] | <0.001 |
| Resp Rate | 0.65 | [0.54-0.75] | 0.01 |
| Glucose | 0.53 | [0.43-0.64] | 0.47 |
| Platelet Ct | 0.57 | [0.46-0.68] | 0.17 |
| BMI² | 0.57 | [0.46-0.67] | 0.22 |

The AUC and P values were generated using Graph pad Prism 5.0 software.

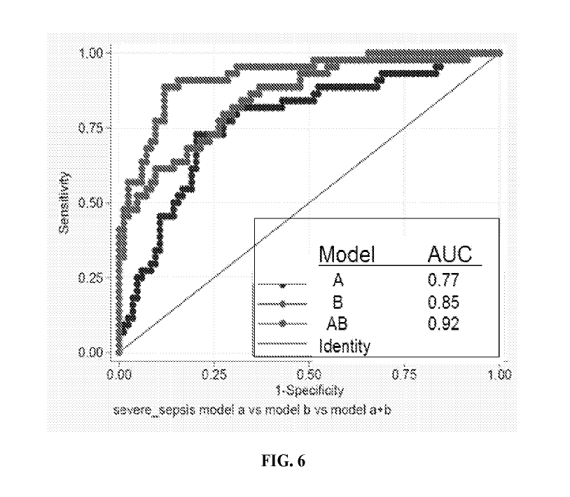

Diagnostic utility logistic regression models were used to predict severe sepsis/septic shock (FIG. 6). The strength of Models A, B, and AB were assessed (table below).

Strength of the Models to Predict Sepsis

| | |
|---|---|
| | Early/No |
| Sepsis vs. | Sepsis vs. Severe |
| No Sepsis | Sepsis and Shock |

| | AUC* | P Value | AUC* | AIC |
|---|---|---|---|---|
| Model A | 0.78 | P < 0.001 | 0.77 | 158 |
| Model B | 0.83 | P < 0.001 | 0.85 | 123 |
| Model AB | 0.90 | P < 0.001 | 0.92 | 114 |

*P < 0.001

Conclusions.

Concentrations of IL-6, IL-10, TNF, CRP, and LBP were individually significantly different among patients with sepsis, severe sepsis, septic shock and SIRS. Logistic regression models combining five inflammatory biomarkers, demographics, vital signs and/or laboratory values better predicted sepsis, severe sepsis or septic shock compared to single markers alone in the patients. The combined model (AB) is contemplated to have clinical utility to more-accurately predict those who have or will develop sepsis, severe sepsis and shock among ED patients presenting with SIRS or another sepsis risk factors.

### Example 6

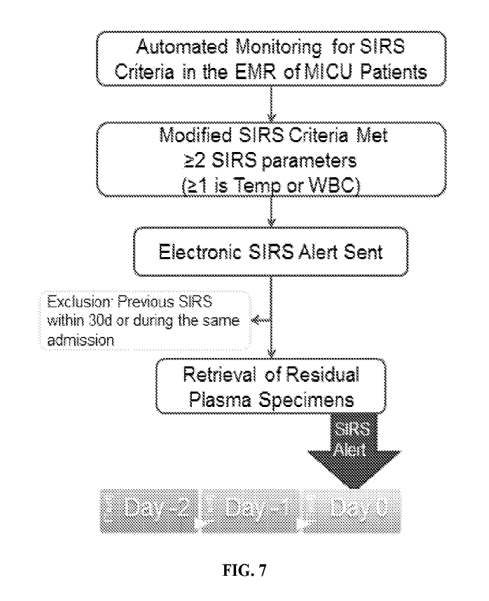

Studies were conducted, which included patients in the medical intensive care unit (MICU). Patients were identified as described in FIG. 7. Residual plasma specimens were collected on the day of SIRS and up to two days prior (FIG. 2). Specimens were stored at −80° C. until just prior to analysis. Stability studies were performed for the analytes studied.

Materials and Methods.

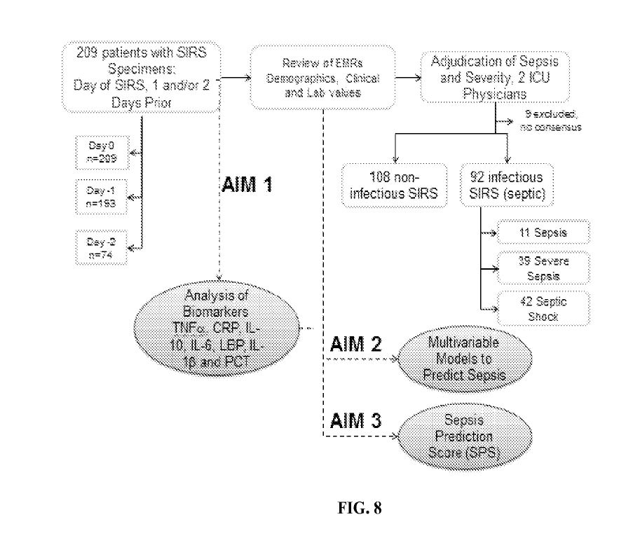

The experimental design is outlined in FIG. 8. TNFα, IL-6, IL-10, CRP, LBP and IL-1b were quantified by immunoassays on the Immulite 1000 (Siemens Healthcare Diagnostics). PCT was quantified using the B•R•A•H•M•S® PCT assay on the VIDAS® analyzer (Biomeriéux).

Statistical Analyses.

Logistic Regression Models:

Logistic regression models (Models A, B, B′, AB, and AB′, described below) were tested using Stata Software (StataCorp). Akaike information criterion (AIC) was used to measure the goodness of fit of the models.

Sepsis Prediction Scores (SPSs):

The probability of developing sepsis or shock was estimated by regressing these outcomes against biomarker concentrations follows:

Logistic Regression Equation

Raw Score=b0 (intercept)+b1 (conc of marker 1)+b2 (conc of marker 2) . . . bn (conc of marker n)

Conversion into a Sepsis Score

$$Sepsis Prediction Score ( SPS ) = \frac{{e}^{Raw Score}}{1 + {e}^{Raw Score}} * 100$$

ROC Analyses:

Generated AUCs for individual biomarkers and SPSs using GraphPad Prism (GraphPad Software).

Objectives.

The clinical utility of early sepsis prediction models to identify sepsis among critically-ill patients with SIRS was investigated through the following aims: to study the clinical utility of inflammatory biomarkers to predict early sepsis; to develop multivariate analyses to predict early sepsis; to investigate the clinical utility of sepsis prediction score (SPSs).

Results.

Baseline Demographics

| Demographics | SIRS | Sepsis | p-value |
|---|---|---|---|
| Total, n (%) | 108 (54%) | 92 (46%) | |
| Female | 51% | 57% | n.s. |
| Male | 49% | 43% | |
| Age (years), mean ± SD | | | |
| Female | 54 ± 16 | 56 ± 16 | n.s. |
| Male | 57 ± 18 | 60 ± 17 | |
| Race | | | |
| Caucasian | 77% | 78% | n.s. |
| African American | 17% | 15% | |
| Other | 6% | 7% | |

Diagnostic performance of inflammatory biomarkers for the
prediction of sepsis up to two days before SIRS and on the
day of SIRS.

| | Day −2 | | Day −1 | | Day 0 | |
|---|---|---|---|---|---|---|
| Biomarker | AUC | p-value | AUC | p-value | AUC | p-value |
| PCT | 0.68 | 0.01 | 0.66 | 0.0002 | 0.73 | <0.0001 |
| TNF-a | 0.58 | 0.26 | 0.60 | 0.02 | 0.64 | 0.0008 |
| IL1b | 0.58 | 0.23 | 0.60 | 0.01 | 0.64 | 0.001 |
| IL-6 | 0.76 | 0.0001 | 0.70 | <0.0001 | 0.73 | <0.0001 |
| IL-10 | 0.70 | 0.004 | 0.64 | 0.0006 | 0.66 | 0.0001 |
| LBP | 0.72 | 0.001 | 0.71 | <0.0001 | 0.74 | <0.0001 |
| CRP | 0.77 | <0.0001 | 0.70 | <0.0001 | 0.73 | <0.0001 |

The following logistic regression models were investigated for their ability to predict early sepsis and shock.

| | | Model AB |
|---|---|---|
| Model A: Baseline | Model B and B′ | and AB′ |
| (1) Clinical and Lab Values | Model B: 5 Biomarkers | Combination |
| White Blood Count | TNFα | of A and B |
| Respiratory Rate | IL-6 | Combination |
| Morning Blood | IL-10 | of A and B′ |
| Glucose | CRP | |
| Heart Rate | LBP | |
| Temperature | Model B′: 7 Biomarkers | |
| Platelets | TNFα | |
| (2) Demographics | IL-6 | |
| Age | IL-10 | |
| Gender | CRP | |
| Body Mass Index | LBP | |
| | IL1β | |
| | PCT | |

Clinical utility of regression models for the risk of developing sepsis

| | Day −2 | | | Day −1 | | | Day 0 | | |
|---|---|---|---|---|---|---|---|---|---|
| Model | ROC | p-value | AIC | ROC | p-value | AIC | ROC | p-value | AIC |
| A | 0.78 | 0.04 | 140 | 0.75 | 0.0002 | 222 | 0.79 | <0.00001 | 214 |
| B | 0.85 | <0.00001 | 79 | 0.74 | <0.00001 | 235 | 0.76 | <0.00001 | 230 |
| B′ | 0.86 | 0.0001 | 81 | 0.75 | <0.00001 | 238 | 0.77 | <0.00001 | 232 |
| AB | 0.94 | 0.0001 | 71 | 0.79 | <0.00001 | 203 | 0.84 | <0.00001 | 188 |
| AB′ | 0.94 | 0.0002 | 73 | 0.80 | <0.00001 | 206 | 0.85 | <0.00001 | 190 |

Clinical utility of regression models for the risk of developing septic shock

| | Day −2 | | | Day −1 | | | Day 0 | | |
|---|---|---|---|---|---|---|---|---|---|
| Model | ROC | p-value | AIC | ROC | p-value | AIC | ROC | p-value | AIC |
| A | 0.78 | 0.1566 | 94 | 0.79 | 0.0029 | 145 | 0.80 | <0.00001 | 146 |
| B | 0.85 | <0.00001 | 50 | 0.80 | <0.00001 | 140 | 0.83 | <0.00001 | 126 |
| B′ | 0.87 | 0.0001 | 52 | 0.81 | <0.00001 | 144 | 0.84 | <0.00001 | 129 |
| AB | 0.93 | 0.0001 | 6 | 0.85 | <0.00001 | 125 | 0.90 | <0.00001 | 114 |
| AB′ | 0.94 | 0.0002 | 26 | 0.85 | <0.00001 | 128 | 0.91 | <0.00001 | 116 |

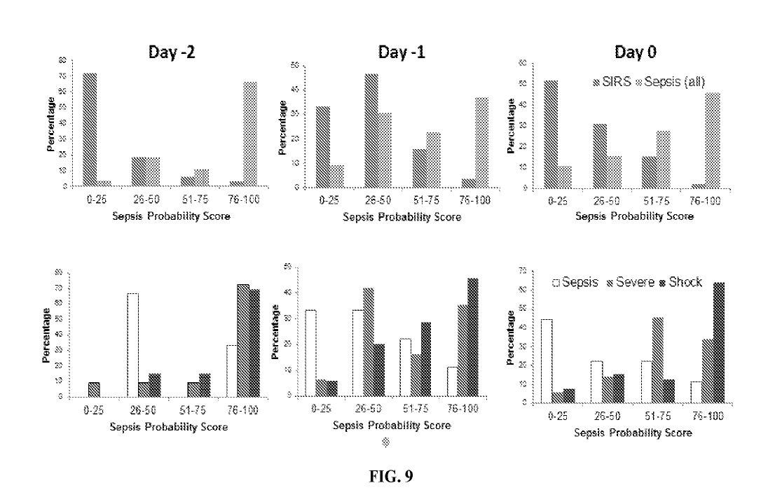

With reference to FIG. 9, SPSs derived from Model AB are useful for risk stratification. Patients with SIRS and sepsis were grouped according to their SPS into quartiles; upper diagrams (FIG. 9). The lower diagrams (FIG. 9) represent patient stratified by sepsis severity and grouped in the corresponding SPS quartile.

Clinical performance of the SPSs from model
AB for the risk of developing sepsis

| | Day −2 | Day −1 | Day 0 |
|---|---|---|---|
| Cut-off: | >25 | >25 | >25 |
| Sens (%) | 89 | 91 | 96 |
| Spec (%) | 53 | 34 | 69 |
| PPV (%) | 49 | 42 | 61 |
| NPV (%) | 90 | 88 | 97 |
| LR+ | 1.89 | 1.38 | 3.10 |
| LR− | 0.21 | 0.26 | 0.06 |

Conclusions

Individual biomarkers have limited clinical utility for the prediction of sepsis and septic shock on the day of SIRS and before SIRS. Logistic regression models combining inflammatory biomarkers, demographic, clinical and common laboratory values better predicted sepsis and septic shock on the day of SIRS than any single marker. These models may also be of value one or two days prior to diagnosis of SIRS. PCT did not improve the regression models in these studies. Utilization of SPSs for early diagnosis of sepsis and risk stratification makes these models interpretable to clinicians. Implementation in clinical practice is contemplated, and it is contemplated that SPSs may facilitate closer scrutiny of patients with SIRS and faster diagnosis and initiation of therapy for the septic patient.

### Example 7

### Exemplary Sepsis Prediction Model

Biomarker data—A panel of 5 biomarkers (Table 1A) are proposed for being expressed early in the sepsis process were measured on the Immulite 1000 automated immunoassay platform in patients up to 2 days prior to developing systemic inflammatory response syndrome (SIRS) in the medical ICU. Extensive chart review was performed to collect demographics (e.g., as in Table 2A) as well as basic laboratory values (e.g., as in Table 2B).

Sepsis Prediction Model Assembly

Three separate logistic regression models were generated using STATA software. For each logistic regression model, a coefficient (weighting characteristic) for each variable and one model constant (alpha) was determined. The formula for each model is as follows:

log(odds of developing sepsis)=log(probability of developing sepsis/probability of not developing sepsis)=alpha+(coef var1)*var1+(coef var 2)*var2+ . . .

The formula is based on the results for parameters selected from Tables 1 and 2 for patients with and without sepsis (about 200 patients). The formula can be used to determine the probably of an individual patient developing sepsis by determining an amount in a sample from the subject of each biomarker in a biomarker panel, determining the clinical parameters about the subject, and calculating a risk ratio using the formula.

Model #1 is a logistical regression model using biomarkers from Table 1A weighting according to the coefficients generated in the analysis. Model #2 is a logistical regression formula which incorporates demographic variables (Table 2A) and basic laboratory values (Table 2B).

Model #3 combines the regression analyses of model #1 and model #2. Using these models, the probability for sepsis was generated for each patient. ROC curves were derived using the linear predictor from each model (Delong, 1988). Cut-offs for a positive and negative risk score were determined at pre-determined (e.g., optimal) sensitivity and specificity on the ROC curve.

The clinical utility for such a sepsis prediction model would be to input cytokine, demographics, and basic laboratory values (Tables 1A, 2A, 2B) into a software program containing the logistical regression formula (s) and have the program calculate the sepsis risk score. Previous studies have utilized such logistical regression models to predict risk of ovarian cancer (Moore, 2008; Moore, 2009). The sepsis risk score could help triage patients in the ED or direct treatment decisions for ICU patients.

All publications, patents, and patent applications mentioned in this specification are herein incorporated by reference to the same extent as if each individual publication, patent, or patent application was specifically and individually indicated to be incorporated by reference. Such references include those set forth in the following list:

### REFERENCES

Andaluz-Ojeda D, Bobillo F, Iglesias V, et al. A combined score of pro- and anti-inflammatory interleukins improves mortality prediction in severe sepsis. Cytokine. 2012; 57(3):332-336.
Angus, D. C., W. T. Linde-Zwirble, et al. (2001). “Epidemiology of severe sepsis in the United States: analysis of incidence, outcome, and associated costs of care.” Crit Care Med 29(7): 1303-10.
Arnold R C, Shapiro N I, Jones A E, et al. Multicenter study of early lactate clearance as a determinant of survival in patients with presumed sepsis. Shock. 2009; 32(1):35-39.
Bone, R. C., R. A. Balk, et al. (1992). “Definitions for sepsis and organ failure and guidelines for the use of innovative therapies in sepsis. The ACCP/SCOM Consensus Conference Committee. American College of Chest Physicians/Society of Critical Care Medicine.” Chest 101(6):1644-55.
Colón-Franco J M and Woodworth A. (2013). “Biomarker panels for the diagnosis of sepsis,” Clinical Laboratory International February/March 2013, pp. 26-28.
Dellinger, R P, et al. (2012). “Surviving Sepsis Campaign: International Guidelines for Management of Severe Sepsis and Septic Shock, 2012”. Intensive Care Medicine. 2013; 39(2): 165-228.
Dellinger, R. P., et al. (2013). “Surviving Sepsis Campaign: International Guidelines for Management of Severe and Septic Shock: 2012,” Clinical Care Medicine Journal, 41 (2), pp. 580-637.
Dellinger R P, Carlet, J M, Masur H, et al. Surviving sepsis campaign: international guidelines for management of severe sepsis and septic shock. Crit Care Med. 2004; 32(3):858-873.
Delong E R, Delong D M, Clarkepearson D I. Comparing the Areas under 2 or More Correlated Receiver Operating Characteristic Curves—a Nonparametric Approach. Biometrics. 1988; 44(3):837-45.
DeMarco M L, Ford B A. Beyond identification: emerging and future uses for MALDI-TOF mass spectrometry in the clinical microbiology laboratory. Clin Lab Med. 2013; 33(3):611-628.
Faix J D. Biomarkers of sepsis. Crit Rev Clin Lab Sci. 2013; 50(1):23-36.
Gibot S, Bene M C, Noel R, et al. Combination biomarkers to diagnose sepsis in the critically ill patient. Am J Respir Crit Care Med. 2012; 186(1):65-71.
Hall M J, Williams S, Defrances C J, et al. Inpatient care for septicemia or sepsis: a challenge for patients and hospitals. NCHS Data Brief. 2011; (62):1-8.
Hooper M H, Weavind L, Wheeler A P, et al. Randomized trial of automated, electronic monitoring to facilitate early detection of sepsis in the intensive care unit. Crit Care Med. 2012; 40(7):2096-2101.
Hotchkiss, R. S. and I. E. Karl (2003). “The pathophysiology and treatment of sepsis.” N Engl J Med 348(2): 138-50.
Kofoed K, Anderson O, Kronborg G, et al. Use of plasma C-reactive protein, procalcitonin, neutrophils, macrophage migration inhibitory factor, soluble urokinase-type plasminogen activator receptor, and soluble triggering receptor expressed on myeloid cells-1 in combination to diagnose infections: a prospective study. Crit Care. 2007; 11(2):R38.
Kumar, A, D. Roberts, et al. (2006). “Duration of hypotension before initiation of effective antimicrobial therapy is the critical determinant of survival in human septic shode Crit Care Med 34(6): 1589-96.
Levy M M, Bernard G R, Francois B, et al. 2001 SCCM/ESICM/ACCP/ATS/SIS International sepsis definitions conference. Crit Care Med. 2003; 31(4):1250-1256.
Levy M M. The surviving sepsis campaign: results of an international guideline-based performance improvement program targeting severe sepsis. Intensive Care Med. 2010; 36(2):222-231.
Meisner M. In procalcitonin (PCT), A new innovative infection parameter; biochemical and clinical aspects. Stuttgart, New York: Thieme; 2000.
Moore R G et al, The Use of multiple novel tumor biomarkers for detection of ovarian carcinoma in patients with a pelvic mass. Gynecol Oncol. 2008; 108: 402-8.
Moore R G et al. A novel multiple marker bioassay utilizing HE4 and CA125 for the production of ovarian cancer in patients with a pelvic mass. Gynecol Oncol. 2009; 112: 40-6.
Pierrakos C, Vincent J L. Sepsis biomarkers: a review. Crit Care. 2010; 14(1):R15.
Pyle, A. L., Woodworth, A. (2010). “Validation of an 8-cytokind multiplex assay on the Luminex multiplex platform for use in Li-heparin plasma” Poster, MCC 2010 Annual Meeting, Anaheim, Calif.
R Development Core Team (2011). R: A language and environment for statistical computing. R Foundation for Statistical Computing, Vienna, Austria. ISBN 3-900051-07-0, URLhttp:itwww.R-project org/.
Remick D G. Pathophysiology of sepsis. Am J Pathol. 2007; 170(5):1435-1444.
Schuetz P, Raad I, Amin D N. Using procalcitonin-guided algorithms to improve antimicrobial therapy in ICU patients with respiratory infections and sepsis. Curr Opin Crit Care. 2013; 19(5):453-460.
Shapiro N I, Tizeciak S, Hollander J E, et al. A prospective, multicenter derivation of a biomarker panel to assess risk of organ dysfunction, shock, and death in emergency department patients with suspected sepsis. Crit Care Med. 2009; 37(1):96-104.
Simon L, Gauvin f, Amre D K, Saint-Louis P, Lacroix J. Serum procalcitonin and C-reactive protein levels as markers of bacterial infection: a systematic review and meta-analysis. Clin Infect Dis. 2004; 39(2):206-217.
Skvarc M, Stubljar D, Rogina P, et al. Non-culture-based methods to diagnose bloodstream infection: Does it work? Eur J Microbiol Immunol (Bp). 2013; 3(2):97-104.
Soni N J, Galaydick J L, Samson D J, et al. Procalcitonin-guided antibiotic therapy. AHRQ comparative effectiveness review. 2012; 12(8)EHC124-EF.
Tang B M, Eslick G D, Craig J C, McLean A S. Accuracy of procalcitonin for sepsis diagnosis in critically ill patients: systematic review and meta-analysis. Lancet Infect Dis. 2007; 7(3):210-217.

What is claimed is:

1. A method of characterizing sepsis in a subject, comprising:
determining an amount in a biological sample from the subject of each biomarker in a biomarker panel, wherein the biomarker panel comprises at least two of the biomarkers as set forth in Tables 1A, 1B, and 1C; and
calculating a risk index using the amounts of the at least two biomarkers.

2. The method of claim 1, and further comprising determining one or more clinical parameters about the subject, and calculating the risk index using the amounts of the at least two biomarkers and the clinical parameters.

3. The method of claim 2, wherein the risk index is correlated to a predicted clinical outcome.

4. The method of claim 2, wherein calculating the risk index comprises
(a) applying a predetermined weight for the amount of each biomarker in the panel and each of the one or more clinical parameters; and
(b) solving one or more equations, wherein the one or more equations is based, at least in part, on the weights for the biomarker amounts and the clinical parameters.

5. The method of claim 2, wherein the risk index is calculated using at least one algorithm.

6. The method of claim 5, wherein the at least one algorithm comprises a logistic regression analysis.

7. The method of claim 4, wherein the predicted clinical outcome is a diagnosis of sepsis, a diagnosis of early sepsis, or a diagnosis of SIRS.

8. The method of claim 5, wherein the biomarker panel comprises:
(a) TNF-alpha, interleukin-6, interleukin-10, lipopolysaccharide binding protein, and C-reactive protein;
(b) IL-1beta, procalcitonin, absolute neutrophil count, and immature granulocyte count; or
(c) Interleukin-8, GM-CSF, MCP1, and INF-gamma.

9. The method of claim 8, wherein the clinical parameters include demographic parameters and laboratory values.

10. The method of claim 8, wherein the clinical parameters are selected from: respiratory rate, white blood cell count, body temperature, heart rate, systolic blood pressure, morning blood glucose, platelet count, red blood cell count, lactate, PTT, INR, bilirubin, creatinine, urine output, whether the subject is culture positive, location of the positive culture, age at SIRS alert, sex, BMI, origin of patient, APACHE II score, whether the subject received inotropics, whether the subject was receiving antibiotics, whether the subject was on a ventilator, whether the subject was treated with vasopressors, whether the subject was diagnosed with a solid tumor or hematoligical malignancies, whether the subject was receiving immunosuppressive treatment, whether the subject has cirrhosis, whether the subject has chronic renal failure, whether the subject has COPD or Asthma, whether the subject is diabetic, whether the subject was receiving chemotherapy, and whether the subject has HIV/AIDS.

11. The method of claim 1, and further comprising: providing an apparatus capable of detecting the biomarkers in the biomarker panel.

12. The method of claim 1, and further comprising: providing a probe for selectively binding each of the biomarkers in the biomarker panel.

13. The method of claim 12, and further comprising measuring an amount of marker-bound probe for each of the biomarkers.

14. The method of claim 1, wherein the biological sample comprises blood, plasma, or serum.

15. The method of claim 1, wherein the biological sample is extracted.

16. The method of claim 1, comprising determining the amount in the sample of the biomarkers using mass spectrometry (MS) analysis, immunoassay analysis, or both.

17. The method of claim 16, wherein the immunoassay analysis comprises an enzyme-linked immunosorbent assay (ELISA).

18. The method claim 1, and further comprising selecting a treatment or modifying a treatment provided to the subject.

19. The method of claim 1, and further comprising:
determining an amount in each of a series of biological samples sample of each biomarker in the biomarker panel.

20. The method of claim 19, wherein the series of biological samples comprises a first biological sample collected prior to initiation of a prophylaxis or treatment and a second biological sample collected after initiation of the prophylaxis or treatment.

21. A kit, comprising probes for each of the biomarkers in a panel comprising two or more of the biomarkers as set forth in Tables 1A, 1B, and 1C.
